(19) United States
(12) Patent Application Publication
(10) Pub. No.: US 2026/0306797 A1
(43) Pub. Date: Oct. 8, 2026

(54) CATHETERS, SYSTEMS, AND METHODS FOR BYPASSING AN OCCLUSION

(71) Applicant: BARD PERIPHERAL VASCULAR, INC., Franklin Lakes, NJ (US)

(72) Inventors: Sarah L. Degginger, Lakewood, CO (US); Bradley S. Forsyth, Buda, TX (US); Justin Hall, Mesa, AZ (US); Gabriella Romano, Tempe, AZ (US); Kamaldeep Singh, Tempe, AZ (US)

(73) Assignee: BARD PERIPHERAL VASCULAR, INC., Franklin Lakes, NJ (US)

(21) Appl. No.: 19/481,263

(22) PCT Filed: May 9, 2023
(86) PCT No.: PCT/US2023/021493

§ 371 Date: Nov. 4, 2025

(51) Int. Cl. A61M 25/01; A61B 17/00; A61M 25/00

(52) U.S. Cl. CPC A61M 25/0194; A61B 17/00234; A61M 25/0026; A61M 25/007; A61M 25/0108; A61B 2017/00305; A61B 2017/00778; A61B 2017/00876; A61B 2017/00902; A61M 2025/0197; A61M 2205/32; A61M 2205/3331; A61M 2210/12

(57) ABSTRACT

A catheter includes a catheter body and a tip positioned at a distal end of the catheter body. The catheter further includes a first guidewire lumen extending through the catheter body and the tip, where a central axis of a distal opening of the first guidewire lumen is parallel to a longitudinal center line of the catheter. The catheter further includes a second guidewire lumen extending through the catheter body and the tip, where a central axis of a distal opening of the second guidewire lumen is perpendicular to the longitudinal center line of the catheter, and the distal opening of the second guidewire lumen defines an active side of the catheter. The catheter further includes a first magnet positioned along the tip.

### TECHNICAL FIELD

The present disclosure relates to catheters, systems, and methods for bypassing occlusions in the vasculature.

### BACKGROUND

Coronary heart disease may be caused, for example, by atherosclerosis. Atherosclerosis occurs when fat, cholesterol, and/or other substances build up in the walls of blood vessels, forming hard structures called occlusions, e.g., plaques and/or atherosclerotic (stenotic) lesions. Over time, these occlusions may increase in size such that the blood vessels are substantially clogged and/or completely blocked, so as to form a total chronic occlusion (CTO). One method of treating CTOs involves the use of re-entry catheter systems that feature the advancement of guidewires, tools, or other devices from within a lumen of an occluded blood vessel, into the subintimal space of the occluded blood vessel, and back into the lumen of the occluded blood vessel, in order to bypass the occlusion. It may, therefore, be useful to provide re-entry catheter systems that allow for precise positioning and placement of guidewires or other devices around an occlusion in an occluded blood vessel.

### SUMMARY

One challenging aspect of bypassing an occlusion is the ability to accurately maneuver guidewires from the subintimal space of an occluded blood vessel into the true lumen of the occluded blood vessel, and therefore the ability to precisely position guidewires in the vicinity of the occlusion. Accordingly, a need exists for alternative systems, methods, and catheters for bypassing occlusions that ensure guidewires are accurately maneuvered and precisely located in the occluded blood vessel. Embodiments of the present disclosure are directed to improvements over the above limitations by providing catheters including dual guidewire lumens and magnetic tips.

In one embodiment, a catheter includes a catheter body and a tip positioned at a distal end of the catheter body. The catheter further includes a first guidewire lumen extending through the catheter body and the tip, where a central axis of a distal opening of the first guidewire lumen is parallel to a longitudinal center line of the catheter. The catheter further includes a second guidewire lumen extending through the catheter body and the tip, where a central axis of a distal opening of the second guidewire lumen is perpendicular to the longitudinal center line of the catheter, and the distal opening of the second guidewire lumen defines an active side of the catheter. The catheter further includes a first magnet positioned along the tip.

In another embodiment, a system for bypassing an occlusion includes a first catheter. The first catheter includes a first catheter body and a tip positioned at a distal end of the first catheter body. The first catheter further includes a first guidewire lumen extending through the first catheter body and the tip of the first catheter, where a central axis of a distal opening of the first guidewire lumen of the first catheter is parallel to a longitudinal center line of the first catheter. The first catheter further includes a second guidewire lumen extending through the first catheter body and the tip of the first catheter, where a central axis of a distal opening of the second guidewire lumen of the first catheter is perpendicular to the longitudinal center line of the first catheter. The first catheter further includes a first magnet positioned along the tip of the first catheter. The system further includes a second catheter. The first catheter is configured to be positioned in a subintimal space of a blood vessel, and the second catheter is configured to be positioned in a lumen of the blood vessel.

In another embodiment, a method of bypassing an occlusion includes positioning a first catheter in a subintimal space of a blood vessel. The first catheter includes a first catheter body, a tip positioned at a distal end of the first catheter body, and a first guidewire lumen extending through the first catheter body and the tip of the first catheter, where a central axis of a distal opening of the first guidewire lumen of the first catheter is perpendicular to a longitudinal center line of the first catheter. The method further includes positioning a second catheter in a lumen of the blood vessel. The second catheter includes a second catheter body, a tip positioned at a distal end of the second catheter body, and a first guidewire lumen extending through the second catheter body and the tip of the second catheter, wherein a central axis of a distal opening of the first guidewire lumen of the second catheter is perpendicular to a longitudinal center line of the second catheter. The method further includes advancing a guidewire through the first guidewire lumen of the first catheter, through the distal opening of the first guidewire lumen of the first catheter, and into the distal opening of the first guidewire lumen of the second catheter.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a side view of a catheter, according to one or more embodiments shown and described herein;

FIG. 2 schematically depicts a cross section of the catheter of FIG. 1, according to one or more embodiments shown and described herein;

FIG. 3 schematically depicts a frontal view of a distal end of a tip of the catheter of FIG. 1, according to one or more embodiments shown and described herein;

FIG. 4 schematically depicts a bottom view of the active side of the tip of FIG. 1, according to one or more embodiments shown and described herein;

FIG. 5 schematically depicts a side view of a catheter including a force sensor, according to one or more embodiments shown and described herein;

FIG. 6 schematically depicts a cross section of the catheter of FIG. 5, according to one or more embodiments shown and described herein;

FIG. 7 schematically depicts a side view of a catheter including a force sensor and a conductive canal, according to one or more embodiments shown and described herein;

FIG. 8 schematically depicts a side view of a catheter including an optical display and luer connection, according to one or more embodiments shown and described herein;

FIG. 9 schematically depicts a two catheter system where the two catheters are coapted in an occluded blood vessel, according to one or more embodiments shown and described herein; and

FIG. 10 schematically depicts a cross section of the coapted catheters of FIG. 9, according to one or more embodiments shown and described herein.

### DETAILED DESCRIPTION

Embodiments described herein are directed to devices and methods for bypassing an occlusion. In some embodiments, the devices and methods may be used to bypass an occlusion with a guidewire from the subintimal space of a vessel and to re-enter the true lumen of the vessel with the guidewire.

A guidewire may be used to pass one or more treatment devices into the subintimal space of a vessel surrounding an occlusion, and to optionally pass the treatment devices back into the true lumen of the vessel. However, such guidewires may be difficult to precisely position and maneuver from the subintimal space to the true lumen of the vessel, making it difficult to desirably place and deploy an eventual treatment device in the vessel. The embodiments described herein address one or more of the aforementioned limitations. In particular, the devices and methods for bypassing an occlusion described herein may include a catheter having a catheter body and a tip positioned at a distal end of the catheter body. The catheter may further include a first guidewire lumen extending through the catheter body and the tip, where a central axis of a distal opening of the first guidewire lumen is parallel to a longitudinal center line of the catheter. The first guidewire lumen may be used to pass the first catheter over a tracking guidewire in the vessel of interest. The catheter may further include a second guidewire lumen extending through the catheter body and the tip, where a central axis of a distal opening of the second guidewire lumen is perpendicular to the longitudinal center line of the catheter. The catheter may further include a first magnet positioned along the tip configured to mate with a magnet along the tip of a second catheter. The second catheter may similarly include a second guidewire lumen extending through the catheter body and the tip, where a central axis of a distal opening of the second guidewire lumen is perpendicular to the longitudinal center line of the catheter. After the catheters are magnetically aligned and coapted, a re-entry guidewire may be passed through second guidewire lumen of the first catheter and into the second guidewire lumen of the second catheter, thereby allowing accurate placement of the re-entry guidewire through the subintimal lumen of a vessel, as desired. Various embodiments will now be described in greater detail below with reference to the figures.

As used herein, the term “proximal” means closer to or in the direction of an origin of an element, such as a catheter. The origin of a catheter may be a handle or other user-manipulated portion of the catheter. The term “distal” means further from the origin, or handle, of the catheter. Put another way, the term “distal” means closer to or in the direction of a tip of a catheter, which is separated from a handle of the catheter by the length of the catheter body.

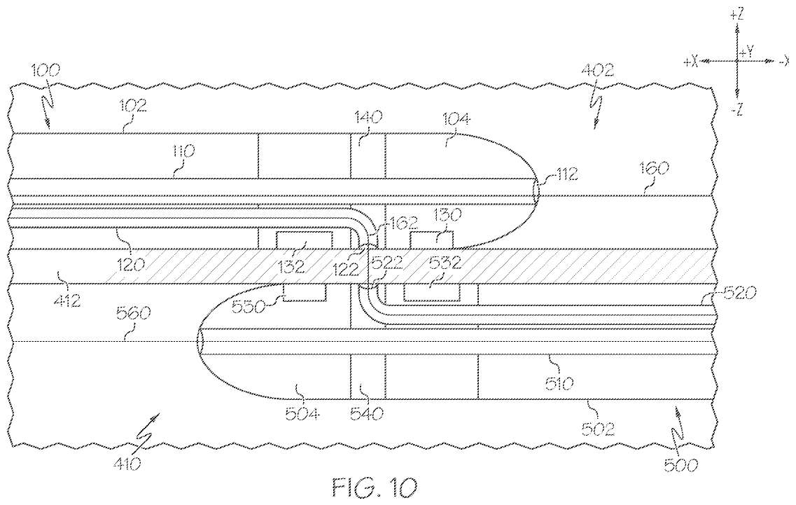

Referring now to FIG. 1, a catheter 100 (e.g., a first catheter) of a system for bypassing an occlusion is depicted. As will be described in greater detail herein, the system may further include a second catheter 500 (FIGS. 9 and 10). While the structure of the catheter 100 will be discussed in detail, it should be appreciated that the structure of the second catheter 500 (FIGS. 9 and 10) may mirror the catheter 100 except where noted. Still referring to FIG. 1, the catheter 100 generally includes a catheter body 102 and a tip 104 that is positioned at a distal end (e.g. in the direction of the −x axis of the coordinate axes of FIG. 1) and particularly configured to aid in advancement of the catheter 100 through a blood vessel. For example, the tip 104 may be pointed and/or atraumatic for advancement through a blood vessel. The catheter body 102 may have any desirable cross-sectional shape and any suitable diameter for intravascular use.

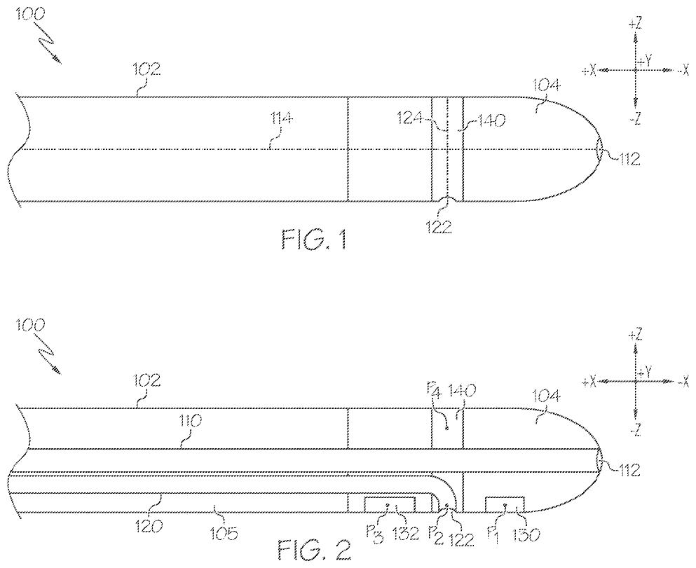

Referring now to FIG. 1 in conjunction with FIG. 2, which depicts a longitudinal cross section of the catheter 100, the catheter 100 further includes a first guidewire lumen 110 and a second guidewire lumen 120 extending at least partially along or through the catheter body 102. For instance, the first guidewire lumen 110 and the second guidewire lumen 120 lumens may extend at least partially longitudinally through the catheter body 102 in the direction of the x-axis of the coordinate axes of FIG. 2.

Referring now to FIGS. 1, 2, and 3, which depicts a frontal view of the tip 104, in embodiments, the first guidewire lumen 110 extends through the length of the catheter body 102 and the tip 104. The first guidewire lumen 110 may include a distal opening 112. The distal opening 112 of the first guidewire lumen 110 may be positioned on distal-most surface of the tip 104. A central axis 114 passing through a geometric center of the distal opening 112 of the first guidewire lumen 110 is parallel to a longitudinal center line (e.g. in the direction of the x-axis of the coordinate axes of FIGS. 1 and 2) of the catheter 100. Particularly, the term “longitudinal center line” as used herein refers to the longitudinal center line of the catheter 100 when the catheter 100 is in a relaxed state. In other words, “longitudinal centerline” refers to the longitudinal center line of the catheter 100 when the catheter 100 is not under any flexions, such as experienced when the catheter 100 is traversed through vasculature, for instance.

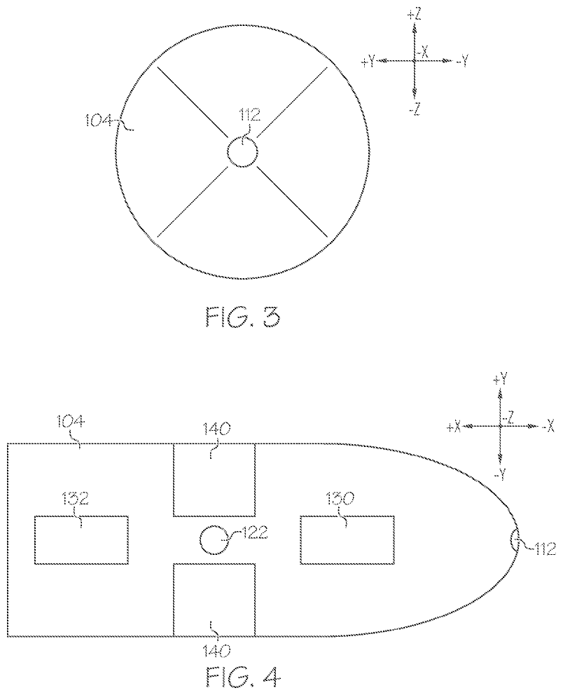

Referring now to FIGS. 1, 2, and 4, which depicts a bottom view of the tip 104, in embodiments, the second guidewire lumen 120 extends through the length of the catheter body 102 and partially through the length of the tip 104. The second guidewire lumen 120 may include a distal opening 122. The distal opening 122 of the second guidewire lumen 120 may be positioned on side surface of the tip 104. The distal opening 122 of the second guidewire lumen 120 may be positioned proximally the distal opening 112 of the first guidewire lumen 110 extend through a sidewall 105 of the catheter body 102. A central axis 124 of the distal opening 122 passing through a geometric center of the second guidewire lumen 120 is perpendicular or otherwise angularly offset from (i.e., not parallel to) the longitudinal center line (e.g. in the direction of the x-axis of the coordinate axes of FIGS. 1 and 2) of the catheter 100. The distal opening 122 of the second guidewire lumen 120 defines an active side of the catheter 100. The active side of the catheter 100 extends across the catheter body 102, the tip 104, and any other elements arranged along the catheter body 102. The active side of the tip 104, is depicted in FIG. 4. The active side of the tip 104, for instance, is the portion of the tip 104 that abuts or faces a vessel wall when a re-entry guidewire 162 (FIGS. 9 and 10) is passed through the vessel wall. For instance, the re-entry guidewire 162 (FIGS. 9 and 10) may protrude from the distal opening 122 of the second guidewire lumen 120 and extend radially away from the longitudinal centerline of the catheter 100 to contact a wall of the blood vessel.

Still referring to FIGS. 1, 2, and 4, in embodiments, the catheter 100 may include one or more magnets 130, 132. In embodiments, the catheter 100 may include a first magnet 130 positioned along the tip 104. In embodiments, the first magnet 130 may be positioned along the active side of the tip 104. In embodiments, the first magnet 130 is positioned distal the distal opening 122 of the second guidewire lumen 120. In embodiments, the first magnet 130 may be positioned such that a lateral center point P₁ of the first magnet 130 is in line with the lateral center point P₂ of the distal opening 122 of the second guidewire lumen 120. In other words, the lateral center point of the first magnet 130 may be in line with the lateral center point of the distal opening 122 of the second guidewire lumen 120 in the direction of the x-axis of the coordinate axes of FIG. 4.

In embodiments, the catheter 100 may include a second magnet 132 in addition to the first magnet 130. In embodiments, the second magnet 132 may be positioned along the active side of the tip 104. In embodiments, the second magnet 132 is positioned proximal the distal opening 122 of the second guidewire lumen 120. In embodiments, the second magnet 132 may be positioned such that a lateral center point P₃ of the second magnet 132 is in line with the lateral center point P₂ of the distal opening 122 of the second guidewire lumen 120. In other words, the lateral center point of the second magnet 132 may be in line with the lateral center point of the distal opening 122 of the second guidewire lumen 120 in the direction of the x-axis of the coordinate axes of FIG. 4. In some embodiments, depending, for example, on the shape of the magnets 130 and 132, the magnets 132 and 134 may generally be aligned with the opening 122, but the center points P₁, P₂ and P₃ may not be aligned. In embodiments, the first magnet 130 and the second magnet 132 may be equally spaced from the distal opening 122 of the second guidewire lumen 120 in the longitudinal direction (e.g. in the direction of the x-axis of FIG. 4).

In embodiments, it should be appreciated that the catheter 100 may include the second magnet 132 without the first magnet 130. That is, the catheter 100 may include the second magnet 132 positioned along the active side of the tip 104 and proximal to the distal opening 122 of the second guidewire lumen 120 without the first magnet 130.

The magnets 130, 132 described herein may be permanent magnets comprising one or more hard magnetic materials, such as but not limited to alloys of rare earth elements (e.g., samarium-cobalt magnets or neodymium magnets, such as N52 magnets) or alnico. In some variations, the magnets 130, 132 may comprise anisotropic magnets; in other variations, the magnets 130, 132 may comprise isotropic magnetics. In some variations, the magnets 130, 132 may be formed from compressed powder. In some variations, a portion of the magnets 130, 132 may comprise one or more soft magnetic materials, such as but not limited to iron, cobalt, nickel, or ferrite. It should be appreciated that in systems comprising two catheters, either the first catheter 100 or the second catheter 500 (FIGS. 9 and 10) may comprise ferromagnetic elements (i.e., elements attracted to but not generating a permanent magnetic field). For example, in some variations, the first catheter 100 may include only one or more ferromagnetic elements while the second catheter 500 (FIGS. 9 and 10) may comprise one or more permanent magnets. In other variations, the second catheter 500 (FIGS. 9 and 10) may include only one or more ferromagnetic elements while the first catheter 100 may comprise one or more permanent magnets. However, in other variations, one or both of the first catheter 100 and the second catheter 500 (FIGS. 9 and 10) may include any suitable combination of ferromagnetic, permanent, and/or other suitable kinds of magnets.

Generally, the dimensions of the magnets 130, 132 described herein may be selected based upon the size of the catheter 100 carrying the magnets 130, 132, which in turn may be selected based upon the anatomical dimensions of the selected blood vessels through which the catheter 100 may be advanced. For example, the catheter 100, at a cross section including the magnets 130, 132 may have a maximum width less than the width or diameter of the anatomical structure the catheter 100 is to be advanced through. The magnets 130, 132 may have any suitable length. In some variations, the magnets 130, 132 may be cuboid magnets. In other embodiments, the magnets 130, 132 may have any suitable shape for placement inside or on the catheter 100. Magnets 130, 132 may be cylindrical, semi-cylindrical, tube-shaped, box-shaped, or the like.

In embodiments, the outer surfaces of the magnets 130, 132 may be flush or in line with the outer surface of the tip 104. In other embodiments, the magnets 130, 132 may be positioned radially within tip 104 away from the outer surface of the 104. Each magnet 130, 132 may be fixed in or on the tip 104 of the catheter 100 by any suitable method. For example, in some variations the magnets 130, 132 may be embedded in, adhered to, or friction-fit within the tip 104.

Still referring to FIGS. 1, 2, and 4, in embodiments, the catheter 100 may include a rotational indicator 140. In embodiments, the rotational indicator 140 may be positioned along the tip 104. The rotational indicator 140 may be made of or otherwise include any material that exhibits high radiopacity, allowing the rotational indicator 140 to be visualized under fluoroscopy. The rotational indicator 140 may have a longitudinal center point P₄ in line with the distal opening 122 of the second guidewire lumen 120. In some embodiments, depending, for example, on the shape of the rotational indicator 140, the rotational indicator 140 may generally be aligned with the opening 122, but the center points P₂ and P₄ may not be aligned. In some embodiments, the rotational indicator 140 may be a continuous band around the circumference of the tip 104 of the catheter 100 that includes one or more discrete markers at one or more known angular and/or longitudinal locations. In such embodiments, the distal opening 122 of the second guidewire lumen 120 may be extend through the rotational indicator 140. In some embodiments, the rotational indicator 140 may be a band that partially extends around the circumference of the tip 104. In such embodiments, the distal opening 122 of the second guidewire lumen 120 may be positioned along the tip 104 within the absence of the rotational indicator 140. In embodiments, the tip 104 may be made of a radiolucent material. In such embodiments, the radiolucent tip 104 can allow energy pass therethrough and the radiopaque rotational indicator 140 may allow for visualization of the position of the tip 104 under fluoroscopy, and more particularly, for visualization of the distal opening 122 of the second guidewire lumen 120, which is in a known position relative the rotational indicator 104, under fluoroscopy.

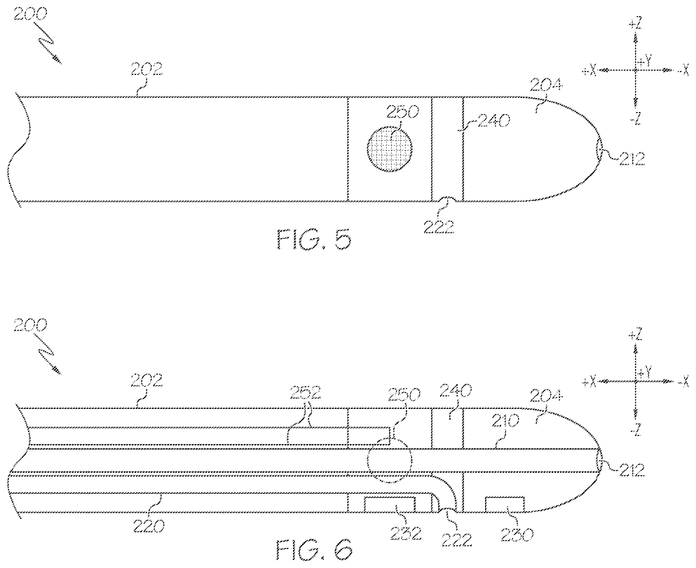

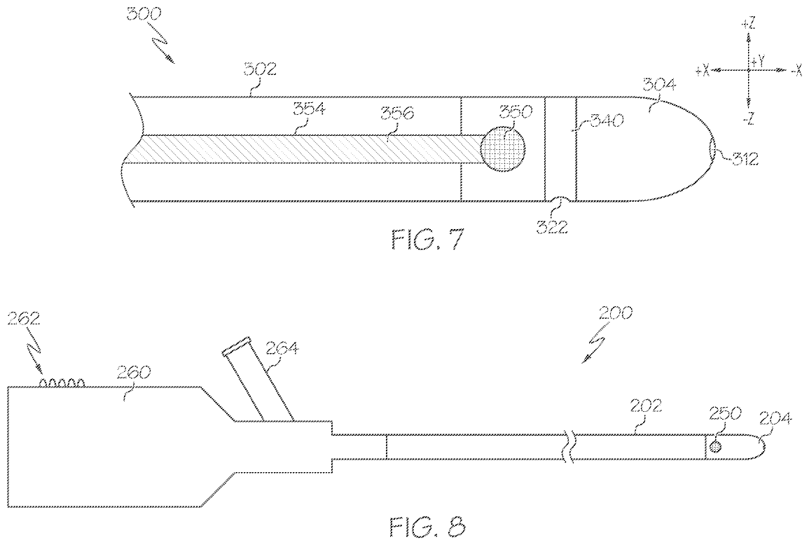

Referring now to FIGS. 5 and 6, a catheter 200 is depicted. The catheter 200 may resemble the catheter 100 previously discussed in all aspects except where noted. For instance, the catheter 200 includes a catheter body 202 having a distal tip 204. The catheter includes a first guidewire lumen 210 having a distal opening 212, and a second guidewire lumen 220 having a distal opening 222. The catheter 200 may further include a first magnet 230 and/or a second magnet 232 positioned along the tip 204. The catheter 200 may further include a rotational indicator 240 positioned along the tip 204. The catheter 200 may further include a force sensor 250. The force sensor 250 may be any suitable sensor, such as a force sensitive resistor. The force sensor 250 may be positioned such that a sensing surface of the force sensor 250 is positioned along a surface of the tip 204. Electrical leads 252 of the force sensor 250 may extend though an interior lumen of the catheter body 202 and be coupled to one or more processors or displays.

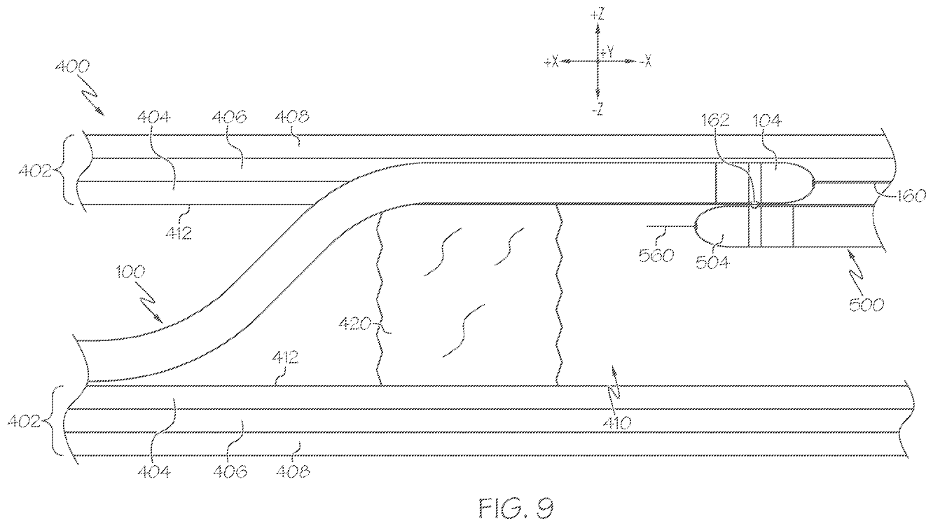

Referring now to FIG. 7, a catheter 300 is depicted. The catheter 300 may resemble the catheters 100 and 200 previously discussed in all aspects except where noted. For instance, the catheter 300 includes a catheter body 302 having a distal tip 304. The catheter 300 includes a first guidewire lumen having a distal opening 312, and a second guidewire lumen having a distal opening 322. The catheter 300 may further include a first magnet and/or a second magnet positioned along the tip 304. The catheter 300 may further include a rotational indicator 340 positioned along the tip 304. The catheter 300 may further include a force sensor 350, similar to the force sensor 250 previously discussed. The force sensor 350 may be positioned such that a sensing surface of the force sensor 350 is positioned along a surface of the tip 304. The catheter 300 may include an etched canal 354 in the surface of the catheter body 302 and the tip 304 of the catheter 300. The etched canal 354 may extend from the force sensor 350 to a proximal end of the catheter 300, and more particularly to one or more processors or displays. A conductive material 356, such as gold tape, may be filled into the etched canal 354, such that the conductive material 356 electrically couples the force sensor 350 to the one or more displays or processors.

Referring now to FIG. 8, the catheter 200 is depicted including a handle 260 at the proximal end of the catheter 200. The handle 260 may include an optical display 262 communicatively coupled to the force sensor 250 through the electrical leads 252 (FIG. 6). The optical display 262 may include one or more screens or lights. In some embodiments, the optical display 262 may provide a numerical display of the force detected by the force sensor 250. In some embodiments, the one or more lights or screens of the optical display 262 may light up in accordance with the force detected by the force sensor 250. For instance, the brightness, color, or number of discrete lights illuminated on the optical display 262 may correlate to the magnitude of the force detected by the force sensor 250. While the catheter 200 has been described as including the optical display 262 coupled to the force sensor 250, it should be appreciated that the catheter 300 discussed with reference to FIG. 7 may include a similar handle and optical display as that discussed with respect to FIG. 8, that in such embodiments, are communicatively coupled with the force sensor 350 (FIG. 7) by the conductive material 356 (FIG. 7).

Still referring to FIG. 8, in embodiments, the catheter 200 may include a luer connection 264 positioned along the handle 260 of the catheter 200. The luer connection 264 may be fluidly coupled with the first guidewire lumen 210 (FIG. 6). During fluoroscopy procedures, a user may introduce a dye or contrast agent into the first guidewire lumen 210 (FIG. 6) from the luer connection 264, which may then be delivered to a treatment site through the distal opening 212 (FIG. 6) of the first guidewire lumen 210 (FIG. 6). While discussed with respect to the catheter 200, it should be appreciated that the catheter 100 (FIG. 1) may include a handle including a luer connector, similar to that discussed with respect to FIG. 8, fluidly coupled to the first guidewire lumen 110 (FIG. 2). Similarly, it should be appreciated that the catheter 300 (FIG. 7) may include a handle including a luer connector, similar to that discussed with respect to FIG. 8, fluidly coupled to the first guidewire lumen (not depicted) of the catheter 300 (FIG. 7).

Referring now to FIGS. 9 and 10, a system and method for bypassing an occlusion 420 within a vessel 400 with the first catheter 100 and the second catheter 500 will now be discussed. It should be appreciated that the first catheter 100 and the second catheter 500 may take the form of any of the embodiments of catheters 100, 200, 300 previously discussed. With respect to the second catheter 500, the second catheter 500 includes a catheter body 502 having a distal tip 504. The second catheter 500 includes a first guidewire lumen 510 having a distal opening 512, and a second guidewire lumen 520 having a distal opening 522. The second catheter 500 may further include a first magnet 530 and/or a second magnet 532 positioned along the tip 504. The second catheter 500 may further include a rotational indicator 540 positioned along the tip 504.

The vessel 400 includes a lumen 410 defined between the endothelium 412 of the vessel 400. The vessel 400 further includes a subintimal space 402 within the wall of the vessel 400. More particularly, the subintimal space 402 includes a tunica intima 404 between the endothelium 412 and the internal elastic membrane of the wall of the vessel 400. The subintimal space 402 further includes a tunica media 406 that may be between the internal elastic membrane of the wall of the vessel 400 and the external elastic membrane of the wall of the vessel 400. The subintimal space further includes a tunica externa 408 forming the outermost layer of the wall of the vessel 400.

Still referring first to FIGS. 9 and 10, the first catheter 100 may be advanced within the lumen 410 of the blood vessel 400 upstream of the occlusion 420. More particularly, the first catheter 100 may track a tracking guidewire 160 through the lumen 410 of the blood vessel 400. The tracking guidewire 160 extends through the first guidewire lumen 110 of the first catheter 100. As the tracking guidewire 160 approaches the occlusion 420, a user may direct the tracking guidewire 160 into the subintimal space 402 of the vessel 400. Therefore, the user may further advance the catheter 100 over the tracking guidewire 160, extending through the first guidewire lumen 110, into the subintimal space 402 of the vessel 400. The user may continue to track the catheter 100 over the tracking guidewire 160 in the subintimal space 402 until the tip 104 of the first catheter 100 is downstream of the occlusion 420.

A user may advance the second catheter 500 within the lumen 410 of the blood vessel 400 from downstream the occlusion 420 toward the occlusion 420. More particularly, the second catheter 500 may track a tracking guidewire 560 through the lumen 410 of the blood vessel 400. The tracking guidewire 560 extends through a first guidewire lumen 510 of the second catheter 500, which resembles the first guidewire lumen 110 of the first catheter 100 previously discussed. As the tracking guidewire 160 approaches the occlusion 420, a user may direct the tracking guidewire 160 into the subintimal space 402 of the vessel 400. Therefore, the user may further advance the catheter 100 over the tracking guidewire 160, extending through the first guidewire lumen 110, into the subintimal space 402 of the vessel 400. The user may continue to track the catheter 100 over the tracking guidewire 160 in the subintimal space 402 until the tip 104 of the first catheter 100 is downstream of the occlusion 420.

The first magnet 130 and the second magnet 132 of the first catheter 100 and the first magnet 530 and the second magnet 532 of the second catheter 500 may be configured to promote rotational and axial alignment of the catheters 100 and 500. Proper axial and rotational alignment between catheters 100 and 500 may facilitate alignment of the distal opening 122 of the second guidewire lumen 120 of the first catheter 100 and the distal opening 522 of the second guidewire lumen 520 of the second catheter 500.

In embodiments, the magnetic material of the magnets 130, 132 of the first catheter 100 may be arranged such that the magnetic field generated by the magnets 130, 132 is stronger in the direction of the active side of the catheter 100 (e.g. in the −z direction of the coordinate axes of FIGS. 9 and 10) than in the direction of the non-active side of the catheter 100 (e.g. in the +z direction of the coordinate axes of FIGS. 9 and 10). Similarly, the magnetic material of the magnets 530, 532 may be arranged such that the magnetic field generated by the magnets 530, 532 is stronger in the direction of the active side of the second catheter 500 (e.g. in the +z direction of the coordinate axes of FIGS. 9 and 10) than in the direction of the non-active side of the second catheter 500 (e.g. in the −z direction of the coordinate axes of FIGS. 9 and 10). In such embodiments, the strength of the magnetic fields in the directions of the active sides of the catheters 100, 500, respectively, and polarity of the magnetic fields may promote rotational alignment between the active side of the first catheter 100 in the subintimal space 402 and the active side of the second catheter 500 in the lumen 410 of the vessel 400. Moreover, the strength of the magnetic fields in the directions of the active sides of the catheters 100, 500, respectively, and polarity of the magnetic fields may promote rotational alignment between the distal opening 122 of the second guidewire lumen 120 of the first catheter 100 and the distal opening 522 of the second guidewire lumen 520 of the second catheter 500.

Each magnet 130, 132 of the first catheter 100 may be configured to mate with a corresponding magnet 530, 532 of the second catheter 500, and vice versa, such that the first catheter 100 and the second catheter 500 may be aligned and coapted. As used herein, the terms “coapted” and/or “strong coaptation” may be understood to mean that the first catheter 100 and the second catheter 500 are in close approximation (e.g. in the direction of the z-axis of the coordinate axes of FIGS. 9 and 10) such that a re-entry guidewire 162 may pass from the distal opening 122 of the second guidewire lumen 120 of the first catheter 100 into the distal opening 522 of the second guidewire lumen 520 of the second catheter 500. For instance, the second magnet 132 positioned a first distance proximal (e.g. in the +x direction of the coordinate axes of FIGS. 9, 10) of the distal opening 122 of the second guidewire lumen 120 of the catheter 100 may be configured to mate with the first magnet 530 positioned the first distance distal (e.g. in the +x direction of the coordinate axes of FIGS. 9, 10) the distal opening 522 of the second guidewire lumen 520 of the second catheter 500. The first magnet 130 positioned a second distance distal (e.g. in the −x direction of the coordinate axes of FIGS. 9, 10) of the distal opening 122 of the second guidewire lumen 120 of the first catheter 100 may be configured to mate with the second magnet 532 positioned the second distance proximal (e.g. in the −x direction of the coordinate axes of FIGS. 9, 10) of the distal opening 522 of the second guidewire lumen 520 of the second catheter 500.

After the first catheter 100 and the second catheter 500 are coapted and aligned, a user may advance the re-entry guidewire 162 through the second guidewire lumen 120 of the first catheter 100. As the re-entry guidewire 162 is advanced, the re-entry guidewire 162 projects from distal opening 122 of the second guidewire lumen 120 and through the endothelium of the vessel 400. Further advancing the re-entry guidewire 162 results in the entry of the re-entry guidewire 162 into the distal opening 522 of the second guidewire lumen 520 of the second catheter 500 and passage of the re-entry guidewire 162 through the second guidewire lumen 520. After placement, the re-entry guidewire 162 therefore provides a track for the user to advance one or more devices, such as stents, balloons, or angipoplasty devices through the subintimal space 402 of the vessel and back into the lumen 410 of the vessel 400, as desired, to treat the occlusion 420. Coaptation and alignment of the first catheter 100 and the second catheter 500 may stretch the endothelium 412 of the vessel 400 into a taut position, which may allow the re-entry guidewire 162 to more easily puncture through the endothelium 412 and re-enter the lumen 410 of the vessel 400.

To further aid a user in determining when the first catheter 100 and the second catheter 500 are aligned and coapted, a user may view the catheters 100, 500 under fluoroscopy to view the rotational indicators 140, 540. By visually analyzing the catheters 100, 500, and particularly the rotational indicators 140, 540, a user may determine when the rotational indicators 140, 540 are in line with each other, which due to the positioning of the rotational indicators 140, 540 along the catheters 100, 500, respectively, further indicates that the distal opening 122 of the second guidewire lumen 120 and the distal opening 522 of the second guidewire lumen 520 are aligned and coapted.

Moreover, in embodiments, to further aid a user in determining the position of the first catheter 100 and/or the second catheter 500 relative the occlusion 420, the user may analyze the feedback from the force sensors 250, 350 (FIGS. 5-7). For instance, in embodiments where the first catheter 100 includes a force sensor, force readings may gradually increase as the tip 104 of the catheter 100 approach the occlusion 420, maximize when the tip 104 is at the position of the occlusion 420, and gradually decrease as the tip 104 begins to further advance past the occlusion 420. Therefore, a user may be able to accurately determine that the tip 104 of the catheter 100 has bypassed the occlusion 420 in the subintimal space 402.

While methods have been described where the first catheter is advanced from a position upstream of the occlusion 420 to a position downstream of the occlusion 420, it should be appreciated that the catheter 100 may similarly be advanced from a position downstream of the occlusion 420, through the subintimal space 402, to a position upstream of the occlusion 420. In such embodiments, it should further be appreciated, that the second catheter 500 may be advanced toward the occlusion 420 from upstream of the occlusion 420.

Embodiments can be described with reference to the following numerical clause:
Clause 1: A catheter, comprising: a catheter body; a tip positioned at a distal end of the catheter body; a first guidewire lumen extending through the catheter body and the tip, wherein a first central axis of a first distal opening of the first guidewire lumen is parallel to a longitudinal center line of the catheter; a second guidewire lumen extending through the catheter body and the tip, wherein: a second central axis of a second distal opening of the second guidewire lumen is angularly offset from the longitudinal center line of the catheter; and the second distal opening of the second guidewire lumen is at an active side of the catheter; and a first magnet positioned along the tip.
Clause 2: The catheter of clause 1, wherein the second central axis of the second distal opening of the second guidewire lumen is perpendicular to the longitudinal center line of the catheter.
Clause 3: The catheter of clause 1 or 2, wherein the first magnet is positioned distal the second distal opening of the second guidewire lumen.
Clause 4: The catheter of any of 1 or 2, wherein the first magnet is positioned proximal the second distal opening of the second guidewire lumen.
Clause 5: The catheter of any of clauses 1-3, further comprising a second magnet positioned along the tip, wherein: the first magnet is positioned distal the second distal opening of the second guidewire lumen; and the second magnet is positioned proximal the second distal opening of the second guidewire lumen.
Clause 6: The catheter of any of clauses 1-5, further comprising a rotational indicator positioned along the tip.
Clause 7: The catheter of clause 6, wherein: the tip is radiolucent; and the rotational indicator is radiopaque.
Clause 8: The catheter of any of clauses 1-7, further comprising a pressure sensor positioned along a surface of the tip.
Clause 9: A system for bypassing an occlusion, comprising: a first catheter, comprising: a first catheter body; a first tip positioned at a first distal end of the first catheter body; a first guidewire lumen extending through the first catheter body and first the tip of the first catheter, wherein a first central axis of a first distal opening of the first guidewire lumen of the first catheter is parallel to a first longitudinal center line of the first catheter; a second guidewire lumen extending through the first catheter body and the first tip of the first catheter, wherein a second central axis of a second distal opening of the second guidewire lumen of the first catheter is angularly offset from the first longitudinal center line of the first catheter; and a first magnet positioned along the tip of the first catheter; and a second catheter, wherein: the first catheter is configured to be positioned in a subintimal space of a blood vessel; and the second catheter is configured to be positioned in a lumen of the blood vessel.
Clause 10: The system of clause 9, wherein the second catheter comprises: a second catheter body; a second tip positioned at a second distal end of the second catheter body; a third guidewire lumen extending through the second catheter body and the second tip of the second catheter, wherein a third central axis of a third distal opening of the third guidewire lumen of the second catheter is parallel to a second longitudinal center line of the second catheter; a fourth guidewire lumen extending through the second catheter body and the second tip of the second catheter, wherein a fourth central axis of a fourth distal opening of the second guidewire lumen of the second catheter is angularly offset from the second longitudinal center line of the second catheter; and a second magnet positioned along the tip of the second catheter.
Clause 11: The system of clause 10, wherein: the second distal opening of the second guidewire lumen of the first catheter defines a first active side of the first catheter; the fourth distal opening of the second guidewire lumen of the second catheter defines a second active side of the second catheter; the first magnet of the first catheter is positioned along the first active side of the first tip of the first catheter; and the second magnet of the second catheter is positioned along the second active side of the second tip of the second catheter.
Clause 12: The system of clause 11, wherein the first magnet of the first catheter is configured to magnetically mate with the second magnet of the second catheter when the first catheter is positioned in the subintimal space of the blood vessel, and the second catheter is positioned in the lumen of the blood vessel.
Clause 13: The system of clause 12, wherein: the first catheter further comprises a first rotational indicator positioned along the first tip of the first catheter; the second catheter further comprises a second rotational indicator positioned along the second tip of the second catheter; and the first rotational indicator of the first catheter and the second rotational indicator of the second catheter are arranged and configured to be aligned when the first magnet of the first catheter is magnetically mated with the second magnet of the second catheter.
Clause 14: The system of clause 12 or 13, wherein the second distal opening of the second guidewire lumen of the first catheter and the fourth distal opening of the second guidewire lumen of the second catheter are arranged and configured to be aligned when the first magnet of the first catheter is magnetically mated with the second magnet of the second catheter.
Clause 15: A method of bypassing an occlusion, comprising: positioning a first catheter in a subintimal space of a blood vessel, wherein the first catheter comprises: a first catheter body; a first tip positioned at a first distal end of the first catheter body; and a first guidewire lumen extending through the first catheter body and the first tip of the first catheter, wherein a first central axis of a first distal opening of the first guidewire lumen of the first catheter is angularly offset from a first longitudinal center line of the first catheter; positioning a second catheter in a lumen of the blood vessel, wherein the second catheter comprises: a second catheter body; a second tip positioned at a second distal end of the second catheter body; and a second guidewire lumen extending through the second catheter body and the second tip of the second catheter, wherein a second central axis of a second distal opening of the second guidewire lumen of the second catheter is angularly offset from a second longitudinal center line of the second catheter; and advancing a guidewire through the first guidewire lumen of the first catheter, through the first distal opening of the first guidewire lumen of the first catheter, and into the second distal opening of the second guidewire lumen of the second catheter.
Clause 16: The method of claim 15, further comprising: advancing the first catheter to a position in the subintimal space of the blood vessel to bypass the occlusion from a first direction; and advancing the second catheter to a position in the lumen of the blood vessel to bypass the occlusion from a second direction, wherein the second direction is opposite the first direction.
Clause 17: The method of clause 16, further comprising: advancing the first catheter through the subintimal space of the blood vessel from a first point upstream of the occlusion to the position in the subintimal space of the blood vessel to bypass the occlusion, wherein the position in the subintimal space of the blood vessel to bypass the occlusion is downstream of the occlusion.
Clause 18: The method of any of clauses 15-17, further comprising magnetically mating the first catheter and the second catheter, wherein: the first catheter further comprises a first magnet positioned along the first tip of the first catheter; the second catheter further comprises a second magnet positioned along the second tip of the second catheter; and the first distal opening of the first guidewire lumen of the first catheter and the second distal opening of the second guidewire lumen of the second catheter are arranged and configured to be aligned when the first catheter and the second catheter are magnetically mated.
Clause 19: The method of any of clauses 15-18, further comprising: tracking the first catheter over a first guidewire to a position in the subintimal space of the blood vessel to bypass the occlusion, wherein the first catheter further comprises a third guidewire lumen configured to track the first guidewire, wherein: the third guidewire lumen of the first catheter extends through the first catheter body and the tip of the first catheter; and a third central axis of a third distal opening of the third guidewire lumen of the first catheter is parallel to the first longitudinal center line of the first catheter; and tracking the second catheter over a second guidewire to a position in the lumen of the blood vessel to bypass the occlusion, wherein the second catheter further comprises a fourth guidewire lumen configured to track the second guidewire, wherein: the fourth guidewire lumen of the second catheter extends through the second catheter body and the second tip of the second catheter; and a fourth central axis of a fourth distal opening of the second guidewire lumen of the second catheter is parallel to the second longitudinal center line of the second catheter.
Clause 20: The method of any of clauses 15-19, wherein: the guidewire is advanced from the first distal opening of the first guidewire lumen of the first catheter, through an endothelium of the blood vessel, and into the second distal opening of the second guidewire lumen of the second catheter.

It should now be understood that embodiments of the present disclosure are directed to devices, systems, and methods for bypassing an occlusion in a vessel. In particular, the devices and methods for bypassing an occlusion described herein may include a catheter having a catheter body and a tip positioned at a distal end of the catheter body. The catheter may further include a first guidewire lumen extending through the catheter body and the tip, where a central axis of a distal opening of the first guidewire lumen is parallel to a longitudinal center line of the catheter. The first guidewire lumen may be used to pass the first catheter over a tracking guidewire in the vessel of interest. The catheter may further include a second guidewire lumen extending through the catheter body and the tip, where a central axis of a distal opening of the second guidewire lumen is perpendicular to the longitudinal center line of the catheter. The catheter may further include a first magnet positioned along the tip configured to mate with a magnet along the tip of a second catheter. The second catheter may similarly include a second guidewire lumen extending through the catheter body and the tip, where a central axis of a distal opening of the second guidewire lumen is perpendicular to the longitudinal center line of the catheter. After the catheters are magnetically aligned and coapted, a re-entry guidewire may be passed through second guidewire lumen of the first catheter and into the second guidewire lumen of the second catheter, thereby allowing accurate placement of the re-entry guidewire through the subintimal lumen of a vessel, as desired.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter, comprising:
a catheter body;
a tip positioned at a distal end of the catheter body;
a first guidewire lumen extending through the catheter body and the tip, wherein a first central axis of a first distal opening of the first guidewire lumen is parallel to a longitudinal center line of the catheter;
a second guidewire lumen extending through the catheter body and the tip, wherein:
a second central axis of a second distal opening of the second guidewire lumen is angularly offset from the longitudinal center line of the catheter; and
the second distal opening of the second guidewire lumen is at an active side of the catheter; and
a first magnet positioned along the tip.

2. The catheter of claim 1, wherein the second central axis of the second distal opening of the second guidewire lumen is perpendicular to the longitudinal center line of the catheter.

3. The catheter of claim 1, wherein the first magnet is positioned distal the second distal opening of the second guidewire lumen.

4. The catheter of claim 1, wherein the first magnet is positioned proximal the second distal opening of the second guidewire lumen.

5. The catheter of claim 1, further comprising a second magnet positioned along the tip, wherein:
the first magnet is positioned distal the second distal opening of the second guidewire lumen; and
the second magnet is positioned proximal the second distal opening of the second guidewire lumen.

6. The catheter of claim 1, further comprising a rotational indicator positioned along the tip.

7. The catheter of claim 6, wherein:
the rotational indicator is radiopaque.

8. The catheter of claim 1, further comprising a pressure sensor positioned along a surface of the tip.

9. A system for bypassing an occlusion, comprising:
a first catheter, comprising:
a first catheter body;
a first tip positioned at a first distal end of the first catheter body;
a first guidewire lumen extending through the first catheter body and first the tip of the first catheter, wherein a first central axis of a first distal opening of the first guidewire lumen of the first catheter is parallel to a first longitudinal center line of the first catheter;
a second guidewire lumen extending through the first catheter body and the first tip of the first catheter, wherein a second central axis of a second distal opening of the second guidewire lumen of the first catheter is angularly offset from the first longitudinal center line of the first catheter; and
a first magnet positioned along the tip of the first catheter; and
a second catheter, wherein:
the first catheter is configured to be positioned in a subintimal space of a blood vessel; and
the second catheter is configured to be positioned in a lumen of the blood vessel.

10. The system of claim 9, wherein the second catheter comprises:
a second catheter body;
a second tip positioned at a second distal end of the second catheter body;
a third guidewire lumen extending through the second catheter body and the second tip of the second catheter, wherein a third central axis of a third distal opening of the third guidewire lumen of the second catheter is parallel to a second longitudinal center line of the second catheter;
a fourth guidewire lumen extending through the second catheter body and the second tip of the second catheter, wherein a fourth central axis of a fourth distal opening of the second guidewire lumen of the second catheter is angularly offset from the second longitudinal center line of the second catheter; and
a second magnet positioned along the tip of the second catheter.

11. The system of claim 10, wherein:
the second distal opening of the second guidewire lumen of the first catheter defines a first active side of the first catheter;
the fourth distal opening of the second guidewire lumen of the second catheter defines a second active side of the second catheter;
the first magnet of the first catheter is positioned along the first active side of the first tip of the first catheter; and
the second magnet of the second catheter is positioned along the second active side of the second tip of the second catheter.

12. The system of claim 11, wherein the first magnet of the first catheter is configured to magnetically mate with the second magnet of the second catheter when the first catheter is positioned in the subintimal space of the blood vessel, and the second catheter is positioned in the lumen of the blood vessel.

13. The system of claim 12, wherein:
the first catheter further comprises a first rotational indicator positioned along the first tip of the first catheter;
the second catheter further comprises a second rotational indicator positioned along the second tip of the second catheter; and
the first rotational indicator of the first catheter and the second rotational indicator of the second catheter are arranged and configured to be aligned when the first magnet of the first catheter is magnetically mated with the second magnet of the second catheter.

14. The system of claim 12, wherein the second distal opening of the second guidewire lumen of the first catheter and the fourth distal opening of the second guidewire lumen of the second catheter are arranged and configured to be aligned when the first magnet of the first catheter is magnetically mated with the second magnet of the second catheter.

15. A method of bypassing an occlusion, comprising:
positioning a first catheter in a subintimal space of a blood vessel, wherein the first catheter comprises:
a first catheter body;
a first tip positioned at a first distal end of the first catheter body; and
a first guidewire lumen extending through the first catheter body and the first tip of the first catheter, wherein a first central axis of a first distal opening of the first guidewire lumen of the first catheter is angularly offset from a first longitudinal center line of the first catheter;
positioning a second catheter in a lumen of the blood vessel, wherein the second catheter comprises:
a second catheter body;
a second tip positioned at a second distal end of the second catheter body; and
a second guidewire lumen extending through the second catheter body and the second tip of the second catheter, wherein a second central axis of a second distal opening of the second guidewire lumen of the second catheter is angularly offset from a second longitudinal center line of the second catheter; and
advancing a guidewire through the first guidewire lumen of the first catheter, through the first distal opening of the first guidewire lumen of the first catheter, and into the second distal opening of the second guidewire lumen of the second catheter.

16. The method of claim 15, further comprising:
advancing the first catheter to a position in the subintimal space of the blood vessel to bypass the occlusion from a first direction; and
advancing the second catheter to a position in the lumen of the blood vessel to bypass the occlusion from a second direction, wherein the second direction is opposite the first direction.

17. The method of claim 16, further comprising:
advancing the first catheter through the subintimal space of the blood vessel from a first point upstream of the occlusion to the position in the subintimal space of the blood vessel to bypass the occlusion, wherein the position in the subintimal space of the blood vessel to bypass the occlusion is downstream of the occlusion.

18. The method of claim 15, further comprising magnetically mating the first catheter and the second catheter, wherein:
the first catheter further comprises a first magnet positioned along the first tip of the first catheter;
the second catheter further comprises a second magnet positioned along the second tip of the second catheter; and
the first distal opening of the first guidewire lumen of the first catheter and the second distal opening of the second guidewire lumen of the second catheter are arranged and configured to be aligned when the first catheter and the second catheter are magnetically mated.

19. The method of claim 15, further comprising:
tracking the first catheter over a first guidewire to a position in the subintimal space of the blood vessel to bypass the occlusion, wherein the first catheter further comprises a third guidewire lumen configured to track the first guidewire, wherein:
the third guidewire lumen of the first catheter extends through the first catheter body and the tip of the first catheter; and
a third central axis of a third distal opening of the third guidewire lumen of the first catheter is parallel to the first longitudinal center line of the first catheter; and
tracking the second catheter over a second guidewire to a position in the lumen of the blood vessel to bypass the occlusion, wherein the second catheter further comprises a fourth guidewire lumen configured to track the second guidewire, wherein:
the fourth guidewire lumen of the second catheter extends through the second catheter body and the second tip of the second catheter; and
a fourth central axis of a fourth distal opening of the second guidewire lumen of the second catheter is parallel to the second longitudinal center line of the second catheter.

20. The method of claim 15, wherein:
the guidewire is advanced from the first distal opening of the first guidewire lumen of the first catheter, through an endothelium of the blood vessel, and into the second distal opening of the second guidewire lumen of the second catheter.
